(19) United States
(12) Patent Application Publication
(10) Pub. No.: US 2021/0182992 A1
(43) Pub. Date: Jun. 17, 2021

(54) INTELLIGENT MONITORING CONTROL SYSTEM BASED ON THE ANALYSIS OF PRISONER INFORMATION

(71) Applicant: HEFEI COMPASS ELECTRONIC TECHNOLOGY CO., LTD., Anhui (CN)

(72) Inventors: Ye SHI, Anhui (CN); Liankun DANG, Anhui (CN)

(73) Assignee: HEFEI COMPASS ELECTRONIC TECHNOLOGY CO., LTD., Anhui (CN)

(21) Appl. No.: 16/309,916

(22) PCT Filed: Oct. 31, 2018
(86) PCT No.: PCT/CN2018/112953

§ 371 Date: Dec. 13, 2018

(30) Foreign Application Priority Data
Sep. 21, 2018 (CN) 201811108345.3

(51) Int. Cl. G06Q 50/26; A61B 5/00

(52) U.S. Cl. CPC G06Q 50/26; A61B 5/4815

(57) ABSTRACT

The present application discloses an intelligent monitoring control system based on the analysis of prisoner information. Model building module is configured to establish a identifier-time-age model and store. Information counting module is configured to count the sleep time, the sleep duration, the average sleep time, and the average duration of each prisoner in the prison form days. Information regularization module is configured to determine the abnormality level of the prisoner. Intelligent control module is configured to formulate an early warning strategy based on an analysis result of the imprisonment time and/or age of the prisoner and feed back. The present application compares the sleep situation of each prisoner in the prison with his historical sleep condition to judge whether the prisoner has potential danger.

This application claims priority to Chinese Patent Application No. 201811108345.3, filed to the Chinese Patent Office on Sep. 21, 2018, entitled “Intelligent monitoring control system based on the analysis of prisoner information”, the entire disclosure of which is incorporated herein by reference.

### TECHNICAL FIELD

The present application relates to the technical field of monitoring control methods, and in particular to intelligent monitoring control system based on the analysis of prisoner information.

### BACKGROUND OF THE INVENTION

With the gradual improvement of the prison management system and the continuous updating of prison administration facilities, the ability of prisons to prevent and control various security incidents has been greatly enhanced. However, as the types of crimes and the composition of the prisoners become more complex, the retaliatory, murderous and deceitful nature of the offenders is enhanced, under the influence of impetuous psychology and restlessness, it is so little careless that they will take risks and take the opportunity to take hostages, escape, and commit suicide, which has extremely adverse effects on the personal safety of the prison guards and the continued stability of the place. In order to solve the above problems, it is necessary to conduct a comprehensive and careful detection and analysis of the state of the prisoners in the prison, to ensure the effectiveness of the prison monitoring system and ensure the safety of prison personnel monitoring.

### SUMMARY OF THE INVENTION

Based on the technical problems existing in the background art, the present application proposes an intelligent monitoring control system based on the analysis of prisoner information.

The intelligent monitoring control system based on the analysis of prisoner information proposed in the present application, includes:

model building module, configured to assign an imprisonment identifier to each prisoner in a prison, and establish an identifier-time-age model based on a correspondence between each of the imprisonment identifier and an imprisonment time and the age of the corresponding prisoner, and store the identifier-time-age model;

information counting module, configured to count a sleep time and a sleep duration of each prisoner in the prison for m days, and calculate an average sleep time and an average duration of each prisoner in the prison for m days;

information regularization module, configured to determine an abnormality level of the prisoner according to respective comparisons between a sleep time of the previous day of the prisoner in the prison and the average sleep time and a sleep duration of the previous day and the average sleep duration;

intelligent control module, configured to acquire an imprisonment time and/or an age of the prisoner in the identification-time-age model according to the abnormality level, formulate an early warning strategy based on an analysis result of the imprisonment time and/or the age of the prisoner, and feed the above warning strategy and the imprisonment identifier of the prisoner back to a supervision department;

wherein m is a preset value and m≥3.

In a preferred technical solution, the information counting module is specifically configured to:

acquire the sleep time of each prisoner in the prison for continuous m days, respectively recorded as t₁, t₂, t₃ . . . tₘ;

calculate the average sleep time of each prisoner for continuous m days according to the following formula, which recorded as t₀, the formula is:

t₀=(t₁+t₂+t₃+ . . . +tₘ−tₘₐₓ−tₘᵢₙ)/(m−2).

wherein, tₘₐₓ=MAX(t₁, t₂, t₃ . . . tₘ), tₘᵢₙ=t₂, t₃ . . . tₘ);

acquire the sleep duration of each prisoner in the prison for continuous m days, respectively recorded as L₁, L₂, L₃ . . . Lₘ;

calculate the average sleep duration of each prisoner for continuous m days according to the following formula, which recorded as L₀, the formula is:

L₀=(L₁+L₂+L₃+ . . . +Lₘ−Lₘₐₓ−Lₘᵢₙ)/(m−2),

wherein, Lₘₐₓ=MAX(L₁, L₂, L₃ . . . Lₘ), Lₘᵢₙ=MIN(L₁, L₂, L₃ . . . Lₘ).

In a preferred technical solution, a preset time difference t_{y} and a preset time difference value L_{y} are stored in the information regularization module;

the information regularization module is specifically configured to:

acquire the sleep time and the sleep duration of the day before the prisoner in the prison, respectively recorded as tₓ, Lₓ;

calculate the difference between tₓ and t₀, Lₓ and L₀, respectively recorded as tₓ₀, Lₓ₀;

compare tₓ₀ with ty, Lₓ₀ and L_{y} respectively:

determining that the prisoner is at a first abnormal level, when tₓ₀<at_{y} or tₓ₀>bt_{y};

determining that the prisoner is at a second abnormal level, when Lₓ0<cL_{y} or Lₓ₀>dL_{y};

determining that the person is at a third abnormal level, when tₓ₀<at_{y} or tₓ₀>bt_{y}, and Lₓ₀<cL_{y} or Lₓ₀>dL_{y};

wherein, a, b, c, and d are preset values, 0<a<1, b>1, 0<c<1, d>1.

In a preferred technical solution, a preset time interval and a preset age range are stored in the intelligent control module;

the intelligent control module is specifically configured to:

acquire an abnormal level of the prisoner;

obtaining the imprisonment time of the prisoner in the identifier-time-age model, when the prisoner is at the first abnormal level;

obtaining the age of the prisoner in the identifier-time-age model, when the prisoner is at the second abnormal level;

obtaining the imprisonment time and the age of the prisoner in the identifier-time-age model, when the prisoner is at the third abnormal level;

calculating the actual time interval between the imprisonment time of the above-mentioned prisoner and the current time;

comparing the above actual time interval with a preset time interval, the age of the prisoner and a preset age range respectively:

formulating a first early warning strategy, when the actual time interval is greater than the preset time interval;

formulating a second early warning strategy, when the age of the prisoner exceeds the preset age range;

formulating a third early warning strategy, when the actual time interval is greater than the preset time interval and the age of the prisoner exceeds the preset age range;

formulating a fourth early warning strategy, when the actual time interval is greater than the preset time interval and the age of the prisoner is within the preset age range;

wherein, the first early warning strategy is to mark the information that the imprisonment time of the prisoner has a longer prison time;

wherein the second early warning strategy is to mark the information that the age of the prisoner exceeds the preset age range;

wherein the third early warning strategy is to mark the information that the imprisonment time of the prisoner has the longer time and the age exceeds the preset age range;

wherein the fourth early warning strategy is to mark the information that the imprisonment time of the prisoner has the longer time and the age of the prisoner is within the preset age range.

In a preferred technical solution, the intelligent control module is further configured to: select different information feedback frequencies according to different early warning strategies;

when formulating the first early warning strategy, select a frequency P1 to feed information back to the supervision department;

when formulating the second early warning strategy, select a frequency P2 to feed information back to the supervision department;

When formulating the third early warning strategy, select a frequency P3 to feed information back to the supervision department;

When formulating the fourth early warning strategy, select a frequency P4 to feed information back to the supervision department;

wherein, P1, P2, P3 and P4 are preset values, P1<P3, P2<P3, P4<P3.

In a preferred technical solution, in the model building module, in the identifier-time-age model, the imprisonment identifier, the imprisonment time, and the age are one-to-one correspondence.

In a preferred technical solution, in the model building module, when a prisoner has multiple imprisonment times, the first imprisonment time is selected as the imprisonment time corresponding to the imprisonment identifier of the prisoner.

The intelligent monitoring control system based on the analysis of prisoner information proposed in the present application, first, determines the abnormal level of the prisoner by comparing the sleep time of each prisoner with the average sleep time, the sleep duration and the average sleep duration, analyzes the imprisonment time and/or age of the prisoner according to different abnormal levels, selects the imprisonment time and/or the age of the prisoner according to different abnormal levels, to verify whether the anomaly of each prisoner's sleep time and/or sleep duration is related to the imprisonment time and/or the age of the prisoner, and feeds back the results of the analysis to the supervision department in real time.

On the one hand, the supervisory department is made aware of the preliminary judgment results of the system on the abnormal state of the prisoner, and provided an accurate and effective reference to further analyze the actual state of the prisoner. On the other hand, it is beneficial for the supervisory department to know in time that the prisoners may have potential dangers, and facilitates the supervisory department to take regulatory measures against them, in order to ensure the stability and security of the prison environment.

The present application compares the sleep situation of each prisoner in the prison with his historical sleep condition to judge whether the prisoner has potential danger. On the basis of ensuring the validity of the comparison process and the accuracy of the comparison result, it is realized the comprehensive and precise control of the actual state of the prisoners in the prison, to prevent and control the situation, comprehensively and effectively maintaining the stability and security in the prison.

### BRIEF DESCRIPTION OF DRAWING

FIG. 1 is a schematic structural diagram of an intelligent monitoring control system based on the analysis of prisoner information.

### DETAILED DESCRIPTION OF THE INVENTION

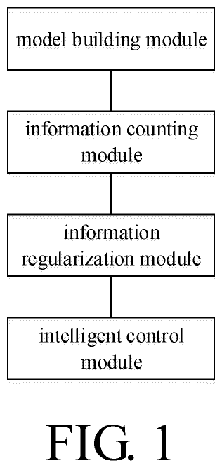

As shown in FIG. 1, FIG. 1 is a schematic structural diagram of an intelligent monitoring control system based on the analysis of prisoner information.

Refer to FIG. 1, the intelligent monitoring control system based on the analysis of prisoner information proposed in the present application includes:

model building module, configured to assign an imprisonment identifier to each prisoner in a prison, and establish an identifier-time-age model based on a correspondence between each imprisonment identifier and an imprisonment time and the age of the corresponding prisoner, and store the identifier-time-age model;

In a present embodiment, in the model building module, in the identifier-time-age model, one imprisonment identifier, one imprisonment time, and one age are one-to-one correspondence.

Further, when a prisoner has multiple imprisonment times, the first imprisonment time is selected as the imprisonment time corresponding to the imprisonment identifier of the prisoner; the first imprisonment time was chosen to understand the starting time of the prisoner to adapt to prison life, and to weaken the impact of the incompatibility of the prisoner on the subsequent analysis results.

Information counting module, configured to count a sleep time and a sleep duration of each prisoner in the prison for m days, and calculate an average sleep time and an average duration of each prisoner in the prison for m days; where, m is a preset value and m≥3.

In a present embodiment, the information counting module is specifically configured to:

acquire the sleep time of each prisoner in the prison for continuous m days, respectively recorded as t₁, t₂, t₃ . . . tₘ;

calculate the average sleep time of each prisoner for continuous m days according to the following formula, which recorded as t₀, the formula is:

t₀=(t₁+t₂+t₃+ . . . +tₘ−tₘₐₓ−tₘᵢₙ)/(m−2);

wherein tₘₐₓ=MAX(t₁, t₂, t₃ . . . tₘ), tₘᵢₙ=t₂, t₃ . . . tₘ);

acquire the sleep duration of each prisoner in the prison for continuous m days, respectively recorded as L₁, L₂, L₃ . . . Lₘ;

calculate the average sleep duration of each prisoner for continuous m days according to the following formula, which recorded as L₀, the formula is:

L₀=(L₁+L₂+L₃+ . . . +Lₘ−Lₘₐₓ−Lₘᵢₙ)/(m−2);
wherein Lₘₐₓ=MAX(L₁, L₂, L₃ . . . Lₘ), Lₘᵢₙ=L₂, L₃ . . . Lₘ).

Information regularization module, configured to determine an abnormality level of the prisoner according to respective comparisons between a sleep time of the previous day of the prisoner in the prison and the average sleep time and a sleep duration of the previous day and the average sleep duration.

In a present embodiment, a preset time difference t_{y} and a preset time difference value L_{y} are stored in the information regularization module.

The information regularization module is specifically configured to:

acquire the sleep time and the sleep duration of the day before the prisoner in the prison, respectively recorded as tₓ, Lₓ;

calculate the difference between tₓ and to, Lₓ and L₀, respectively recorded as tₓ₀, Lₓ₀;

compare tₓ₀ with ty, Lₓ₀ and L_{y} respectively:

when tₓ₀<at_{y} or tₓ₀>bt_{y}, it indicates that the prisoner's sleep time on the previous day was too early or too late, that is, the prisoner's sleep time on the previous day is abnormal, which is marked as the abnormal state, determining that the prisoner is at the first abnormal level;

when Lₓ₀<cL_{y} or Lₓ₀>dL_{y}, it indicates that the sleep duration of the prisoner was too short or too long, that is, the sleep duration of the prisoner was abnormal for the previous day, determining that the prisoner is at the second abnormal level.

when tₓ₀<at_{y} or tₓ₀>bt_{y}, Lₓ₀<cL_{y} or Lₓ₀>dL_{y}, it indicates that the prisoner's sleep time on the previous day was too early or too late, and the sleep duration on the previous day was too short or too long, that is, the sleep time and the sleep duration of the prisoner are abnormal on the previous day, which is marked as the abnormal state, determining that the person is at the third abnormal level;

where, a, b, c, and d are preset values, 0<a<1, b>1, 0<c<1, d>1.

Intelligent control module, configured to acquire an imprisonment time and/or an age of the prisoner in the identification-time-age model according to the abnormality level, formulate an early warning strategy based on an analysis result of the imprisonment time and/or the age of the prisoner, and feed the above warning strategy and the imprisonment identifier of the prisoner back to a supervision department.

In a present embodiment, a preset time interval and a preset age range are stored in the intelligent control module.

The intelligent control module is specifically configured to:

acquire an abnormal level of the prisoner;

when the prisoner is at the first abnormal level, that is, the prisoner's sleep time on the previous day was abnormal, then obtaining the imprisonment time of the prisoner in the identifier-time-age model;

when the prisoner is at the second abnormal level, that is, the prisoner's sleep duration on the previous day was abnormal, then obtaining the age of the prisoner in the identifier-time-age model;

when the prisoner is at the third abnormal level, that is, the sleep time and sleep duration of the prisoner on the previous day both were abnormal, then obtaining the imprisonment time and the age of the prisoner in the identifier-time-age model;

calculating an actual time interval between the imprisonment time of the above-mentioned prisoner and the current time;

respectively comparing the above actual time interval with the preset time interval, the age of the prisoner and the preset age range:

when the actual time interval is greater than the preset time interval, it indicates that the prisoner has a longer prison time, so his possibility of not adapting to prison life is small, that is, the sleep time of the prison on the previous day is not related to the imprisonment time, then formulating a first early warning strategy; the first early warning strategy is to mark the information that the imprisonment time of the prisoner is a longer time; which facilitates the supervisory department timely aware of the situation of the prisoner's abnormal sleep time on the previous day;

when the age of the prisoner exceeds the preset age range, it indicates that the actual age of the prisoner is not within the preset range, that is, it is not common to have a short or excessive sleep duration in the case of his actual age, then formulating a second early warning strategy, to make the supervisory department notices the anomaly; the second early warning strategy is to mark the information that the age of the prisoner exceeds the preset age range;

when the actual time interval is greater than the preset time interval and the age of the prisoner exceeds the preset age range, it indicates that the imprisonment time of the prisoner has less influence on the abnormality of sleep time of the previous day, and the actual age has less influence on the abnormality of sleep duration of the previous day, then formulating a third early warning strategy, to notify the supervisory department to keep abreast of the actual status of the prisoner; the third early warning strategy is to mark the information that the imprisonment time of the prisoner is a longer time and the age exceeds the preset age range;

when the actual time interval is greater than the preset time interval and the age of the prisoner is within the preset age range, it indicates that the imprisonment time of the prisoner has little influence on the abnormality of the previous day's sleep time, and the abnormal sleep duration on the previous day may be due to the influence of his actual age, formulating a fourth early warning strategy, to reduce the misjudgment of the system; the fourth early warning strategy is to mark the information that the imprisonment time of the prisoner is a longer time and the age of the prisoner is within the preset age range.

In a further embodiment, the intelligent control module is further configured to:

select different information feedback frequencies according to different early warning strategies;

when formulating the first early warning strategy, select a frequency P1 to feed information back to the supervision department;

when formulating the second early warning strategy, select a frequency P2 to feed information back to the supervision department;

when formulating the third early warning strategy, select a frequency P3 to feed information back to the supervision department;

when formulating the fourth early warning strategy, select a frequency P4 to feed information back to the supervision department;

wherein, P1, P2, P3 and P4 are preset values, P1<P3, P2<P3, P4<P3.

Selecting different information feedback frequencies is conducive to reflecting the urgency of abnormal situations through the frequency, to facilitate the supervisory department take timely targeted treatment measures and plans according to actual conditions and actual needs, comprehensively ensuring the safety in the prison.

The intelligent monitoring control system based on the analysis of prisoner information proposed in the present application, first, determines the abnormal level of the prisoner by comparing the sleep time of each prisoner with the average sleep time, the sleep duration and the average sleep duration, analyzes the imprisonment time and/or age of the prisoner according to different abnormal levels, selects the imprisonment time and/or the age of the prisoner according to different abnormal levels, to verify whether the anomaly of each prisoner's sleep time and/or sleep duration is related to the imprisonment time and/or the age of the prisoner, and feeds back the results of the analysis to the supervision department in real time.

On one hand, the supervisory department is made aware of the preliminary judgment results of the system on the abnormal state of the prisoner, and provided an accurate and effective reference to further analyze the actual state of the prisoner. On the other hand, it is beneficial for the supervisory department to know in time that the prisoners may have potential dangers, and facilitates the supervisory department to take regulatory measures against them, in order to ensure the stability and security of the prison environment.

The present application compares the sleep situation of each prisoner in the prison with his historical sleep condition to judge whether the prisoner has potential danger. On the basis of ensuring the validity of the comparison process and the accuracy of the comparison result, it is realized the comprehensive and precise control of the actual state of the prisoners in the prison, to prevent and control the situation, comprehensively and effectively maintaining the stability and security in the prison.

The above is only the preferred embodiment of the present application, but the scope of protection of the present application is not limited thereto, and any equivalents or modifications of the technical solutions of the present application and the application concept thereof should be included in the scope of the present application within the scope of the technical scope of the present application.

1. An intelligent monitoring control system based on the analysis of prisoner information, including:
model building module, configured to assign an imprisonment identifier to each prisoner in a prison, and establish an identifier-time-age model based on a correspondence between each imprisonment identifier and an imprisonment time and the age of the corresponding prisoner, and store the identifier-time-age model;
information counting module, configured to count a sleep time and a sleep duration of each prisoner in the prison for m days, and calculate an average sleep time and an average duration of each prisoner in the prison for m days;
information regularization module, configured to determine an abnormality level of the prisoner according to respective comparisons between a sleep time of the previous day of the prisoner in the prison and the average sleep time and a sleep duration of the previous day and the average sleep duration; and
intelligent control module, configured to acquire an imprisonment time and/or an age of the prisoner in the identification-time-age model according to the abnormality level, formulate an early warning strategy based on an analysis result of the imprisonment time and/or the age of the prisoner, and feed the above warning strategy and the imprisonment identifier of the prisoner back to a supervision department;
wherein m is a preset value and m≥3.

2. The intelligent monitoring control system based on the analysis of prisoner information according to claim 1, wherein the information counting module is specifically configured to:
acquire the sleep time of each prisoner in the prison for continuous m days, respectively recorded as t₁, t₂, t₃ . . . tₘ;
calculate the average sleep time of each prisoner for continuous m days according to the following formula, which recorded as t₀, the formula is:
t₀=(t₁+t₂+t₃+ . . . +tₘ−tₘₐₓ−tₘᵢₙ)/(m−2),
wherein tₘₐₓ=MAX(t₁, t₂, t₃ . . . tₘ) and tₘᵢₙ=MIN(t₁, t₂, t₃ . . . tₘ);
acquire the sleep duration of each prisoner in the prison for continuous m days, respectively recorded as L₁, L₂, L₃ . . . Lₘ;
calculate the average sleep duration of each prisoner for continuous m days according to the following formula, which recorded as L₀, the formula is:
L₀=(L₁±L₂±L₃+ . . . +Lₘ−Lₘₐₓ-Lₘᵢₙ)/(m−2),
wherein Lₘₐₓ=MAX(L₁, L₂, L₃ . . . Lₘ) and Lₘᵢₙ=MIN(L₁, L₂, L₃ . . . Lₘ).

3. The intelligent monitoring control system based on the analysis of prisoner information according to claim 2, wherein a preset time difference t_{y} and a preset time difference value L_{y} are stored in the information regularization module;
the information regularization module is specifically configured to:
acquire the sleep time and the sleep duration of the day before the prisoner in the prison, respectively recorded as tₓ, Lₓ;
calculate the difference between tₓ and t₀, Lₓ and L₀, respectively recorded as tₓ₀, Lₓ₀;
compare tₓ₀ with t_{y}, Lₓ₀ and L_{y} respectively, wherein
determining that the prisoner is at a first abnormal level, when tₓ₀<at_{y} or tₓ₀>bt_{y};
determining that the prisoner is at a second abnormal level, when Lₓ₀<cL_{y} or Lₓ₀>dL_{y}; and
determining that the person is at a third abnormal level, when tₓ₀<at_{y} or tₓ₀>bt_{y}, and Lₓ₀<cL_{y} or Lₓ₀>dL_{y},
wherein a, b, c, and d are preset values, and 0<a<1, b>1, 0<c<1, d>1.

4. The intelligent monitoring control system based on the analysis of prisoner information according to claim 3, wherein a preset time interval and a preset age range are stored in the intelligent control module;
the intelligent control module is specifically configured to:
acquire an abnormal level of the prisoner;
obtaining the imprisonment time of the prisoner in the identifier-time-age model, when the prisoner is at the first abnormal level;
obtaining the age of the prisoner in the identifier-time-age model, when the prisoner is at the second abnormal level;
obtaining the imprisonment time and the age of the prisoner in the identifier-time-age model, when the prisoner is at the third abnormal level;
calculating an actual time interval between the imprisonment time of the above-mentioned prisoner and the current time;
respectively comparing the above actual time interval with the preset time interval, the age of the prisoner and the preset age range:
formulating a first early warning strategy, when the actual time interval is greater than the preset time interval;
formulating a second early warning strategy, when the age of the prisoner exceeds the preset age range;
formulating a third early warning strategy, when the actual time interval is greater than the preset time interval and the age of the prisoner exceeds the preset age range; and
formulating a fourth early warning strategy, when the actual time interval is greater than the preset time interval and the age of the prisoner is within the preset age range; wherein,
the first early warning strategy is to mark the information that the imprisonment time of the prisoner has a longer prison time;
the second early warning strategy is to mark the information that the age of the prisoner exceeds the preset age range;
the third early warning strategy is to mark the information that the imprisonment time of the prisoner has the longer time and the age exceeds the preset age range; and
the fourth early warning strategy is to mark the information that the imprisonment time of the prisoner has the longer time and the age of the prisoner is within the preset age range.

5. The intelligent monitoring control system based on the analysis of prisoner information according to claim 4, wherein the intelligent control module is further configured to:
select different information feedback frequencies according to different early warning strategies;
when formulating the first early warning strategy, select a first frequency P1 to feed information back to the supervision department;
when formulating the second early warning strategy, select a second frequency P2 to feed information back to the supervision department;
when formulating the third early warning strategy, select a third frequency P3 to feed information back to the supervision department; and
when formulating the fourth early warning strategy, select a fourth frequency P4 to feed information back to the supervision department;
wherein P1, P2, P3 and P4 are preset values, and P1<P3, P2<P3, P4<P3.

6. The intelligent monitoring control system based on the analysis of prisoner information according to claim 1, wherein in the model building module, in the identifier-time-age model, the imprisonment identifier, the imprisonment time, and the age are one-to-one correspondence.

7. The intelligent monitoring control system based on the analysis of prisoner information according to claim 1, wherein in the model building module, when a prisoner has multiple imprisonment times, the first imprisonment time is selected as the imprisonment time corresponding to the imprisonment identifier of the prisoner.
